(19) United States
(12) Patent Application Publication
(10) Pub. No.: US 2005/0171104 A1
(43) Pub. Date: Aug. 4, 2005

(54) Novel thyroid receptor ligands

(76) Inventors: Mahmoud Rahimi-Ghadim, Stockholm (SE); Neeraj Garg, Tumba (SE); Johan Malm, Trangsund (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 10/510,645

(22) PCT Filed: Feb. 10, 2003
(86) PCT No.: PCT/EP03/01304

(30) Foreign Application Priority Data
Apr. 11, 2002 (GB) 0208384.8

(51) Int. Cl. A61K 31/53; A61K 31/44; A61K 31/4245; A61K 31/421; A61K 31/194

(52) U.S. Cl. 514/241; 514/277; 514/374; 514/378; 514/364; 514/419; 514/569; 544/209; 546/341; 548/132; 548/495; 548/241; 562/466

(57) ABSTRACT

This invention relates to novel compounds according to the general formula: which are thyroid receptor ligands, preferably antagonists, partial antagonists or partial agonists and to methods for using such compounds in the treatment of cardiac and metabolic disorders, such as cardiac arrhythmias, thyro-toxicosis, subclinical hyperthyrodism and liver diseases.

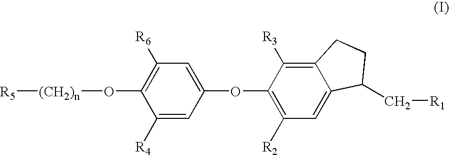

### FIELD OF THE INVENTION

This invention relates to novel compounds which are thyroid receptor ligands, preferably antagonists, and to methods for using such compounds in the treatment of cardiac and metabolic disorders, such as cardiac arrhythmias, thyrotoxicosis, subclinical hyperthyrodism and liver diseases.

### BACKGROUND OF THE INVENTION

Nuclear hormone receptors comprise a class of intracellular, mostly ligand-regulated transcription factors, which include receptors for thyroid hormones. Thyroid hormones exert profound effects on growth, development and homeostasis in mammals. They regulate important genes in intestinal, skeletal and cardiac muscles, liver and the central nervous system, and influence the overall metabolic rate, cholesterol and triglyceride levels, heart rate, and affect mood and overall sense of well being.

There are two major subtypes of the thyroid hormone receptor, TRα and TRβ, expressed from two different genes. Differential RNA processing results in the formation of at least two isoforms from each gene. The TRα₁, TRβ₁ and TRβ₂ isoforms bind thyroid hormone and act as ligand-regulated transcription factors. The TRα₂ isoform is prevalent in the pituitary and other parts of the central nervous system, does not bind thyroid hormones, and acts in many contexts as a transcriptional repressor. In adults, the TRβ₁ isoform is the most prevalent form in most tissues, especially in the liver and muscle. The TRα₁ isoform is also widely distributed, although its levels are generally lower than those of the TRβ₁ isoform. A growing body of data suggest that many or most effects of thyroid hormones on the heart, and in particular on the heart rate and rhythm, are mediated through the TRα₁ isoform, whereas most actions of the hormones on the liver, muscle and other tissues are mediated more through the β-forms of the receptor. It is believed that the α-isoform of the receptor is the major drive to heart rate for the following reasons: (i) tachycardia is very common in the syndrome of generalized resistance to thyroid hormone in which there are defective TRβ-isoforms, and consequently high circulating levels of T₄ and T₃; (ii) Tachycardia was observed in the only described patient with a double deletion of the TRβ gene (Takeda et al, J. Clin. Endrocrinol. & Metab. 1992, 74, 49); (iii) a double knockout TRa gene (but not β-gene) in mice showed bradycardia and lengthening of action potential compared to control mice (Functions of Thyroid Hormone Receptors in Mice: D. Forrest and B. Vennstrom, Thyroid, 2000, 10, 41-52); (iv) western blot analysis of human myocardial TRs show presence of the TRα₁, TRα₂ and TRβ₂ proteins, but not TRβ₁.

If the indications above are correct, an α-selective thyroid hormone receptor antagonist that interacts selectively with the heart would offer an attractive alternative treatment of heart related disorders, such as atrial and ventricular arrhythmias.

Atrial fibrillation (AF) is the most common type of sustained arrhythmia encountered in primary care practice and is significantly more common in elderly patients, thus reflecting a reduction in the threshold for AF with age. Pharmacological treatment of AF involves the following types of anti-arrhythmic drugs according to Vaughan-Williams classification: (i) of class I such as disopyramide and flecainide (sodium channel blocker); (ii) of class III such as amiodarone (potassium channel blocker, prolongation of repolarization); (iii) of class IV such as verapamil and dilitazem (calcium channel blocker). Many patients are also subjected to electric cardioversions in order to convert atrial fibrillation into sinus rhythm. It should be noted that current therapies are associated with pro-arrhythmic risks and anti-arrhythmic agents often have insufficient efficacy partly because effective doses are limited by side-effects.

Ventricular arrhythmia, especially sustained ventricular tachycardia (VT) and ventricular fibrillation (VF) is the main cause of death associated with heart attack. Historically, three types of anti arrhythmic agents, class I agents, β-adrenergic blockers (class II), amiodarone and sotalol, appeared to offer the best scope for mortality reduction in patients with cardiac disease by preventing the occurrence of VT/VF.

The outcome of CAST (Cardiac Arrhythmia Supression Trial, N. Engl. J. Med., 321 (1989) 406-412) and its successor SWORD (Survival With Oral D-sotatol trial, 1994) created much concern regarding the potential of class I agents and sotalol. It was found that class I agents did not decrease mortalities in patient groups at risk for sudden cardiac death. For some subsets of patients, class I agents even proved to increase mortality. The SWORD trial was stopped when sotalol proved to give higher death rate in patients, compared with the placebo. A consequence of these results is that the use of implantable defibrillators and surgical ablation have increased and that the trend in the industry has been towards the development of highly specific class II agents. Some of these channel blockers have been withdrawn from clinical development due to proarrhythmic effects and the subject remains under intensive debate. In this context it should be noted that amiodarone, despite its complex pharmacokinetics, mode of action (amiodarone is not regarded as a pure class III agent) and numerous side effects, is currently considered by many to be the most effective agent in the control of both atrial and ventricular arrhythmia.

Thyrotoxicosis is the clinical syndrome that results when tissues are exposed to elevated levels of circulating thyroid hormones, thyroxine (3,5,3′,5′-tetraiodo-L-thyronine, or T₄) and triiodothyronine (3,5,3′-triiodo-L-thyronine, or T₃). Clinically, this state often manifest itself in weight loss, hypermetabolism, lowering of serum LDL levels, cardiac arrhythmias, heart failure, muscle weakness, bone loss in postmenopausal women, and anxiety. In most instances, thyrotoxicosis is due to hyperthyroidism, a term reserved for disorders characterized by overproduction of thyroid hormones by the thyroid gland. The ideal treatment of hyperthyroidism would be the elimination of its cause. This is however not possible in the more common diseases producing thyroid hypersecretion. At present, treatment of hyperthyroidism is directed to reduce overproduction of thyroid hormones by inhibiting their synthesis or release, or by ablating thyroid tissue with surgery or radioiodine.

Drugs inhibiting thyroid hormone synthesis, release or peripheral conversion of T₄ to T₃ include antithyroid drugs (thionamides), iodide, iodinated contrast agents, potassium perchlorate and glucocorticoids. The main action of antithyroid drugs such as methimazole (MMI), carbimazole, and propylthiouracil (PTU), is to inhibit the organification of iodide and coupling of iodotyrosines, thus blocking the synthesis of thyroid hormones. As they neither inhibit iodide transport or block the release of stored thyroid hormones, control of hyperthyroidism is not immediate and in most cases requires 2 to 6 weeks. Factors that determine the speed of restoration of euthyroidism include disease activity, initial levels of circulating thyroid hormones, and intrathyroidal hormone stores. Serious side effects are not common with antithyroid drugs. Agranulocytosis is the most feared problem and have been observed with both MMI or PTU treatment. Elderly may be more susceptible to this side effect, but agranulocytosis can occur in any age group, although less frequently. Inorganic iodide given in pharmacological doses (as Lugol's solution or as saturated solution of potassium iodide, SSKI) decreases its own transport into the thyroid, thus inhibiting iodide organification (the Wolff-Chaikoff effect), and rapidly blocks the release of T₄ and T₃ from the gland. However, after a few days or weeks, its antithyroid action is lost, and thyrotoxicosis recurs or may worsen. Short-term iodide therapy is used to prepare patients for surgery, usually in combination with a thionamide drug. Iodide is also used in the management of severe thyrotoxicosis (thyroid storm), because of its ability to inhibit thyroid hormone release acutely. Perchlorate interferes with accumulation of iodide by the thyroid. Gastric irritation and toxic reactions limit the long-term use of perchlorate in the management of hyperthyroidism. Glucocorticoids in high doses inhibit the peripheral conversion of T₄ to T₃. In Graves' hyperthyroidism, glucocorticoids appear to decrease T₄ secretion by the thyroid, but the efficiency and duration of this effect is unknown. The aim of surgical treatment or radioiodine therapy of hyperthyroidism is to reduce the excessive secretion of thyroid hormones by removal or destruction of thyroid tissue. Subtotal or near-total thyroidectomy is performed in Graves' disease and toxic multinodular goiter. Restoration of euthyroidism before surgery is mandatory. The classical approach combines a course of thionamide treatment to restore and maintain euthyroidism, and the pre operative administration of iodide for approximately 10 days in order to induce involution of the gland. Propranolol and other beta-adrenergic antagonist drugs are useful in controlling tachycardia and other symptoms of sympathetic activation.

A high affinity ThR antagonist would in principle have the ability to restore euthyrodism quicker than any of the above agents, considered that its action is competitive for the ThR receptor. Such an agent could be used either alone or in combination with above drugs, alternatively before an ablative treatment. It may also serve as a safer substitute for antithyroid drugs, especially in elderly patients at a high risk of agranulocytosis. Furthermore, hyperthyrodism can aggravate pre-existing heart disease and also lead to atrial fibrillation (AF), congestive heart failure, or worsening of angina pectoris. In the elderly patient, often with mild but prolonged elevation of plasma thyroid hormones, symptoms and signs of heart failure and complicating AF may dominate the clinical picture and mask the more classical endocrine manifestations of the disease

### DESCRIPTION OF THE INVENTION

In accordance with the present invention, compounds are provided which are thyroid receptor ligands, having the general formula: 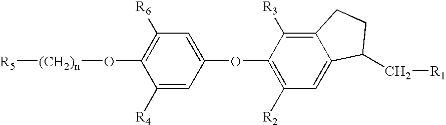
or a pharmaceutically acceptable salt thereof, wherein:
R₁ is independently selected from: carboxylic acid (—CO₂H); phosphonic acid (—PO(OH)₂); phosphamic acid (—PO(OH)NH₂); sulphonic acid (—SO₂OH); hydroxamic acid (—CONHOH); oxamic acid (—NHCOCO₂H); and malonamic acid (—NHCOCH₂CO₂H), or any other possible bioisosteric equivalent of the groups above;
R₂ and R₃ are the same or different and independently selected from: chlorine; bromine; iodide; C₁₋₄ alkyl, said alkyl, or a bioisosteric equivalent optionally substituted with 0, 1, 2 or 3 groups of R^{a} which groups may be the same or different;
R₄ and R₆ are the same or different and independently selected from: hydrogen; halogen; C₁₋₄ alkyl; or a bioisosteric equivalent optionally substituted with 0, 1, 2 or 3 groups of R^{a} which groups may be the same or different;
R₅ is selected from: C₆₋₁₀ aryl; C₁₋₉ heteroaryl, said aryl, and heteroaryl optionally substituted with 0, 1, 2, or 3 groups of R^{b} which groups may be the same or different;
R^{a} represents fluorine or chlorine;
R^{b} represents a member selected from the group of: halogen; —CN; —CO₂H; —CHO; —NH₂; C₁₋₄ alkyl; C₂₋₄ alkenyl; C₂₋₄ alkynyl; C₁₋₄ alkoxy; C₂₋₄ alkenoxy; C₂₋₄ alkynoxy; C₁₋₄ alkylthio; C₂₋₄ alkenylthio; C₂₋₄ alkynylthio; C₆ aryl; C₁₋₅ heteroaryl, C₃₋₆ cycloalkyl; —NH(C₁₋₄); —N(C₁₋₄)₂; —NH(C₆ aryl); —N(C₆ aryl)₂; —NH(C₁₋₅ heteroaryl); and —N(C₁₋₅ heteroaryl)₂ or a bioisosteric equivalent;
n is an integer of 1, 2 or 3;
   included for the variables above are all the possible stereoisomers thereof; prodrug ester forms thereof; and radioactive forms thereof.

### DETAILED DESCRIPTION OF THE INVENTION

The following definitions apply to the terms as used throughout this specification, unless otherwise limited in specific instances. The term “thyroid receptor ligand” as used herein is intended to cover any chemical substance which binds to a thyroid receptor. The ligand may act as an antagonist, a partial antagonist or a partial agonist.

The term “alkyl” as employed herein alone or as part of another group refers to an acyclic straight or branched chain radical, containing 1, 2, 3 or 4 carbons, such as methyl, ethyl, propyl and butyl in the normal chain radical. Alkyl also refer to a radical where 1, 2 or 3 hydrogens can be replaced by halogen through the available carbons. When R₂ and R₃ is selected from alkyl and substituted by halogen, the preferred group radicals is —CF₃, —CHF₂ and —CH₂F.

The term “alkenyl” as used herein by itself or as part of another group refers to straight or branched chain radicals of 2, 3 or 4 carbons and at least one carbon to carbon double bond. Preferably one carbon to carbon double bond is present. Examples of chain radicals is ethenyl, propenyl, 2-methylpropenyl, butenyl and the like. As described above with respect to the “alkyl”, the straight or branched portion of the alkenyl group may be substituted by halogen.

The term “alkynyl” as used herein by itself or as part of another group refers to straight or branched chain radicals of 2 to 4 carbons and at least one carbon to carbon triple bond. Preferably one carbon to carbon triple bond is present. Examples of chain radicals is ethynyl, propynyl, butynyl. As described above with respect to the “alkyl”, the straight or branched portion of the alkenyl group may be substituted by halogen when a substituted alkynyl group is provided.

The term “cycloalkyl” as employed herein alone or as part of another group refers to saturated cyclic hydrocarbon groups or partially unsaturated cyclic hydrocarbon groups, independently containing 1 to 2 carbon to carbon double bonds or carbon to carbon triple bonds. The cyclic hydrocarbon contain 3, 4, 5 or 6 carbons, including rings that are fused. Preferred cycloalkyl groups include 5 or 6 carbons, such as cyclopentyl and cyclohexyl. It should also be understood that the present invention also involve cycloalkyl rings where 1 or 2 carbons in the ring are replaced by either —O—, —S— or —N—, thus forming a saturated or partially saturated heterocycle. Examples of such rings are piperidine, piperazine, morpholine, thiomorpholine, pyrrolidine, oxazolidine, thiazolidine, tetrahydrofurane, tetrahydrothiophene and the like. Preferred heterocyclic rings are 5- or 6-membered, which may be optionally substituted through available carbons as in the case of “aryl” and “heteroaryl”.

The term “aryl” as employed herein alone or as part of another group refers to monocyclic or bicyclic aromatic groups, consisting of 6, 7, 8, 9 or 10 carbons in the ring portion, including partially saturated rings as indanyl and tetrahydronaphthyl. Preferred aryl groups are phenyl and naphthalen, which may be substituted with 0, 1, 2 or 3 groups selected from R^{b} which groups may be the same or different. When R^{b} is selected from C₆ aryl, phenyl is the preferred group.

The term “halogen” as used herein refers to fluorine, chlorine, bromine and iodine. When halogen is selected from R₂ and R₃ the preferred halogen group is bromine or chlorine.

The term “alkoxy”, “alkenoxy” and “alkynoxy” refers to those groups of the designated carbon length in either a straight or branched configuration attached through an oxygen linkage and if two or more carbons in length, they may incude a double or a triple bond. Examples of such alkoxy groups are methoxy, ethoxy, propoxy, allyloxy, propargyloxy, butoxy, tert-butoxy, and the like. Alkoxy also refers to a radical where 1, 2 or 3 hydrogens can be replaced by flourine through the available carbons. When alkoxy and substituted by halogen, the preferred group radicals are —OCF₃, —OCHF₂ and —OCH₂F.

The term “thio” as used herein as a part of another group, exemplified by “alkylthio”, refers to a carbon-sulphur-carbon bond and if two or more carbons in length they may incude a double or a triple bond. The term “thio” may also include higher oxidation states of sulphur, such as sulfoxides —SO— and sulphones —SO₂—. “Alkylthio” also refers to a radical where 1 to 3 hydrogens can be replaced by halogens through the available carbons. When alkylthio is substituted by halogen, the preferred group radicals are —SCF₃, —SCHF₂ and —SCH₂F.

The term “heteroaryl” or “heteroaromatic” as used herein alone or as a part of another group refers to a group containing 1, 2, 3, 4, 5, 6, 7, 8 or 9 carbon atoms, where the aromatic ring includes 1 to 4 heteroatoms, as nitrogen, oxygen or sulphur. Such rings may be fused to another aryl or heteroaryl ring, and includes possible N-oxides. When R₅ is selected from the heteroaryl group may, it may be optionally be substituted by the available carbons with 1 to 3 substituents of R^{b} which groups may be the same or different.

The term “phosphonic acid” and “phosphamic acid” refers to a phosphorus containing group of the structures: 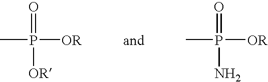
wherein R and R′ are independently selected from hydrogen, C₁₋₄ alkyl, C₂₋₄ alkenyl or C₂₋₄ alkynyl.

The term radical “—N(C₁₋₄)₂” and “—NH(C₁₋₄)” refers to a secondary or tertiary amines where “C” is equal to 1, 2, 3 or 4 carbons in a branched or normal straight chain. Radicals covered by the above definition are: —N(C₁₋₄ alkyl)₂, —NH(C₁₋₄ alkyl), —N(C₂₋₄ alkenyl)₂, —NH(C₂₋₄ alkenyl), —N(C₂₋₄ alkynyl)₂, —NH(C₂₋₄ alkynyl), —N(C₁₋₄ alkyl)(C₂₋₄ alkenyl), —N(C₂₋₄ alkyl)(C₂₋₄ alkynyl), and —N(C₂₋₄ alkenyl)(C₂₋₄ alkynyl).

The term radical “—NH(C₆ aryl)”, “—N(C₆ aryl)₂”, “—NH(C₁₋₃ heteroaryl)” and “—N(C₁₋₅ heteroaryl)₂” refers to secondary or tertiary amines where “C” is equal to a given number of carbon in an aromatic or heteroaromatic ring. The term a “heteroaromatic ring” is defined as above.

The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thia, (iii) hydroxyl vs. amide, (iv) carbonyl vs. oxime, (v) carboxylate vs. tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience: New York, 1970, 64-80; (ii) Burger, A.; “Isosterism and bioisosterism in drug design”; Prog. Drug Res. 1991, 37, 287-371; (iii) Burger A, “Isosterism and bioanalogy in drug design”, Med. Chem. Res. 1994, 4, 89-92; (iv) Clark R D, Ferguson A M, Cramer R D, “Bioisosterism and molecular diversity”, Perspect. Drug Discovery Des. 1998, 9/10/11, 213-224; (v) Koyanagi T, Haga T, “Bioisosterism in agrochemicals”, ACS Symp. Ser. 1995, 584, 15-24; (vi) Kubinyi H, “Molecular similarities. Part 1. Chemical structure and biological activity”, Pharm. Unserer Zeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism: A rational approach in drug design”, Chem. Rev. (Washington, D.C.) 1996, 96, 3147-3176; (ix) Soskic V, Joksimovic J, “Bioisosteric approach in the design of new dopaminergic/serotonergic ligands”, Curr. Med. Chem. 1998, 5, 493-512 (x) Thomber C W, “Isosterism and molecular modification in drug design”, Chem. Soc. Rev. 1979, 8, 563-80.

The compounds of formula I can be present as salts, in particular “pharmaceutically acceptable salts”. A compound having at least one acid group (for example —COOH) can form salts with bases. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono-, di- or tri-lower alkylamine, for example ethyl, tertbutyl, diethyl, diisopropyl, triethyl, tributyl or dimethyl-propylamine, or a mono-, di- or trihydroxy lower alkylamine, for example mono-, di- or triethanolamine. Corresponding internal salts may furthermore be formed. Salts which are unsuitable for pharmaceutical uses but which can be employed, for example, for the isolation or purification of free compounds I or their pharmaceutically acceptable salts, are also included. Preferred salts of the compounds of formula I which include an acid group include sodium, potassium and magnesium salts and pharmaceutically acceptable organic amines.

The compounds of formula I having at least one basic center (for example —NH— in piperidine) can also form acid addition salts. These are formed, for example, with strong inorganic acids, such as mineral acids, for example sulfuric acid, phosphoric acid or a hydrohalic acid, with strong organic carboxylic acids, such as alkanecarboxylic acids of 1 to 4 carbon atoms which are unsubstituted or substituted, for example, by halogen, for example acetic acid, such as saturated or unsaturated dicarboxylic acids, for example oxalic, malonic, succinic, maleic, fumaric, phthalic or terephthalic acid, such as hydroxycarboxylic acids, for example ascorbic, glycolic, lactic, malic, tartaric or citric acid, such as amino acids, (for example aspartic or glutamic acid or lysine or arginine), or benzoic acid, or with organic sulfonic acids, such as (C₁-C₄) alkyl or arylsulfonic acids which are unsubstituted or substituted, for example by halogen, for example methyl- or p-toluenesulfonic acid. Corresponding acid addition salts can also be formed having, if desired, an additionally present basic center. Salts which are unsuitable for pharmaceutical uses but which can be employed, for example, for the isolation or purification of free compounds I or their pharmaceutically acceptable salts, are also included. Preferred salts of the compounds of formula I which include a basic group include monohydrochloride, hydrogensulfate, methanesulfonate, phosphate or nitrate.

An acid center (for example —COOH) part in formula I can form “prodrug ester forms” known in the art such as pivaloyloxymethyl or dioxolenylmethyl. Such prodrug esters are described in standard references such as Chapter 31, written by Camille G. Wermuth et al., in “The Practice of Medicinal Chemistry”, ed. C. G. Wermuth, Academic Press, 1996 (and the references contained therein).

Compounds of the invention can be “stereoisomers”, which have one or more asymmetric centers and can exist in the form of racemates, single enantiomers, as individual diastereomers, with all possible isomers, and mixtures thereof, all of which are within the scope of the invention.

In one embodiment of the present invention there is provided compounds according to formula I, wherein R₅ is carboxylic acid (—CO₂H).

In another embodiment of the present invention there is provided compounds according to formula I and above, wherein R₂ and R₃ is bromine or chlorine.

In yet another embodiment of the present invention there is provided compounds according to formula I and, wherein R₄ is isopropyl and R₆ is hydrogen.

In yet another embodiment of the present invention there is provided compounds according to formula I, wherein R₁ is carboxylic acid (—CO₂H), R₄ is isopropyl and R₆ is hydrogen, and R₂ and R₃ is bromine.

In particularly preferred embodiment of the present invention there is provided compounds according to formula I, said compound being:
{4,6-dibromo-5-[3-isopropyl-4-(naphthalen-2-yl-methoxy)phenoxy]indan-1-yl}acetic acid;
{4,6-dibromo-5-[4-(4-fluorobenzyloxy)-3-isopropylphenoxy]indan-1-yl}acetic acid;
{4,6-dibromo-5-[3-isopropyl-4-(5-methylisoxazol-3-ylmethoxy)phenoxy]indan-1-yl}-acetic acid;
{4,6-dibromo-5-[3-isopropyl-4-(pyridin-2-yl-methoxy)phenoxy]indan-1-yl}acetic acid;
{4,6-dibromo-5-[3-isopropyl-4-(5-phenyl-[1,2,4]oxadiazol-3-ylmethoxy)phenoxy]indan-1-yl}acetic acid;
4-[4-(4,6-dibromo-1-carboxymethyl-indan-5-yloxy)-2-isopropylphenoxymethyl]benzoic acid;
(4,6-dibromo-5-{4-[2-(1H-indol-2-yl)ethoxy]-3-isopropylphenoxy}indan-1-yl)acetic acid;
(4,6-dibromo-5-[3-isopropyl-4-(5-thiophen-3-yl-[1,2,4]oxadiazol-3-yl-methoxy)phenoxy]indan-1-yl}acetic acid;
(5-[4-(4-amino-6-phenylamino[1,3,5]triazin-2-ylmethoxy)-3-isopropylphenoxy]-4,6-dibromoindan-1-yl}acetic acid;
{4,6-dibromo-5-[3-isopropyl-4-(5-methyl-2-phenyloxazol-4-ylmethoxy)phenoxy]indan-1-yl}acetic acid;
{4,6-dibromo-5-[4-(3,5-dimethylisoxazol-4-ylmethoxy)-3-isopropylphenoxy]indan-1-yl}acetic acid;
   and pharmaceutically acceptable salts thereof, and stereoisomers thereof.

The compounds of the invention are antagonists, partial antagonists or partial agonists, preferably α-selective. As such they are useful in medical therapy. Furthermore, they are useful in the prevention, inhibition or treatment of a disease which is dependent on the expression of a T₃ regulated gene or associated with metabolic dysfunction. Examples of such diseases are heart related disorders, such as cardiac arrhytmias (atrial and ventricular arrhythmias), especially atrial fibrillation and ventricular tachyeardia and fibrillation. The compounds of the invention may also be useful for the treatment of thyrotoxicosis, especially in the therapy of elderly patients, subclinical hyperthyroidism, and other related endocrine disorders, related to thyroid hormone.

Compounds of the invention may also be T₃ antagonists with a preferential hepatic activity, and such may be used for medical treatment to improve the clinical course of various liver diseases such as: alcoholic liver disease, viral (Hepatis A,B,C,D,E) liver diseases, and immunological liver diseases. The T₃-antagonist may have principal activity in the liver, and thus have preferential hepatic activity, and with minimal activity in the rest of the body to reduce side-effects associated with the treatment. It is known that induction of a state with abnormally low levels of circulating thyroid hormones (hypothyroidism) is a rewarding treatment of liver diseases as hepatic cirrhosis/fibrosis. Nevertheless, induction of hypothyroidism is not an accepted therapy for liver diseases. The major reason is that currently available methods to induce hypothyroidism inevitably leads to a general hypothyroid state since the thyroid glands production of T₄ is blocked. General, systemic hypothyroidism causes a number of unacceptable clinical symptoms such as myxedema, depression, constipation etc. Also, the time of onset from initiation of therapy until hypothyroidism is manifest is rather long, typically months. T₃-receptor antagonists do also induce hypothyroidism but much faster than standard therapies. A T₃-receptor antagonist with major accumulation in the liver does spare the body from the deleterious impact of general hypothyroidism. The compounds of the invention may therefore be used to treat certain liver diseases, such as chronic alcoholism, acute hepatitis, chronic hepatitis, hepatitis C-induced liver cirrhosis, and liver fibrosis.

The compounds of the invention may also be used to treat certain skin disorders or diseases such as keloids, roughened skin, lichen planus, ichtyosis, acne, psoriasis, Demier's disease, eczema, chloracne, atopic dermatitis, pityriasis, hirsuitism and skin scarring. In treating skin disorders or diseases as described above, the compounds of the invention may be used in combination with a retinoid or a vitamin D analog.

Exemplifying the invention is a pharmaceutical composition comprising any of the compounds described above and a pharmaceutically acceptable carrier. Also exemplifying the invention is a pharmaceutical composition made by combining any of the compounds described above and a pharmaceutically acceptable carrier. An illustration of the invention is a process for making a pharmaceutical composition comprising combining any of the compounds described above and a pharmaceutically acceptable carrier.

Another embodiment of the invention is a method of treating, inhibiting or preventing a disease which is dependent on the expression of a T₃ regulated gene or associated with metabolic dysfunction in a mammal in need thereof by administering to the mammal a therapeutically effective amount of any of the compounds or pharmaceutical compositions described above. The said diseases may be heart related disorders, such as cardiac arrhytmias (atrial and ventricular arrhythmias), especially atrial fibrillation and ventricular tachycardia and fibrillation, especially in the therapy of elderly patients, subclinical hyperthyroidism, and other related endocrine disorders, related to thyroid hormone.

Yet another embodiment of the invention is a method of treating, inhibiting or preventing certain skin disorders or diseases such as keloids, roughened skin, lichen planus, ichtyosis, acne, psoriasis, Demier's disease, eczema, chloracne, atopic dermatitis, pityriasis, hirsuitism and skin scarring. In treating skin disorders or diseases as described above, the compounds of the invention may be used in combination with a retinoid or a vitamin D analog.

Further exemplifying the invention is the use of any of the compounds described above in the preparation of a medicament for the treatment, inhibition or prevention of a disease, which is dependent on the expression of a T₃ regulated gene or associated with metabolic dysfunction. Still further exemplifying the invention is the use of any of the compounds described above in the preparation of a medicament for the treatment and/or prevention of heart related disorders, such as cardiac arrhythmias (atrial and ventricular arrhythmias), especially atrial fibrillation and ventricular tachycardia and fibrillation, especially in the therapy of elderly patients, subclinical hyperthyroidism, and other related endocrine disorders, related to thyroid hormone.

Further exemplifying the invention is the use of any of the compounds described above in the preparation of a medicament for the treatment, inhibition or prevention of certain skin disorders or diseases such as keloids, roughened skin, lichen planus, ichtyosis, acne, psoriasis, Dernier's disease, eczema, chloracne, atopic dermatitis, pityriasis, hirsuitism and skin scarring. In treating skin disorders or diseases as described above, the compounds of the invention may be used in combination with a retinoid or a vitamin D analog.

The compounds of the present invention can be administered in such oral dosage forms as tablets, capsules (each of which includes sustained release or timed release formulations), pills, powder, granules, elixirs, tinctures, suspensions, syrups and emulsions. Likewise, they may also be administered in intravenous (bolus or infusion), intraperitoneal, topical (e.g., ocular eyedrop), subcutaneous, intramuscular, or transdermal (e.g., patch) form, all using forms well known to those of ordinary skill in the pharmaceutical arts.

The dosage regimen utilizing the compounds of the present invention is selected in accordance with a variety of factors including type, species, age, weight, sex, and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 mg per kg of body weight per day (mg/kg/day) to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100, and 500 milligrams of the active ingredient for the symptomatic adjustment of the dosage to the patient to be treated. A medicament typically contains from about 0.01 mg to about 500 mg of the active ingredient, preferably from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches will known to those of ordinary skill in the art. To be administered in the form of a transdermal delivery system, the dosage administration will, of course, be continuous rather than intermittent throughout the dosage regimen.

In the methods of the present invention, the compounds herein described in detail can form the active ingredient, and are typically administered in admixture with suitable pharmaceutical diluents, exipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms includes sodium oleate, sodium stearate, magnesium stearate, sodium benzoate, sodium acetate, sodium chloride and the like. Disintegrators include without limitation starch, methylcellulose, agar, bentonite, xanthan gum and the like.

The compounds of the present invention can also be administered in the form of liposome delivery systems, such as small unilamellar vesicles, large unilamellar vesicles and multilamellar vesicles. Liposomes can be formed form a variety of phospholipids, such as cholesterol, stearylamine or phosphatidylcholines.

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example in “Design of Prodrugs” ed. H. Bundgaard, Elsevier, 1985, which is incorporated by reference herein in its entirety. Metabolites of the compounds includes active species produced upon introduction of compounds of this invention into the biological milieu.

### EXAMPLES

The following Examples represent preferred embodiments of the present invention. However, they should not be construed as limiting the invention in any way. The ¹H NMR spectra was all consistent with the assigned structures in the Examples.

### Example 1

### {4,6-Dibromo-5-[3-isopropyl-4-(naphthalen-2-yl-methoxy)phenoxylindan-1-yl}acetic acid

(a) To a mixture of 5-hydroxy-1-indanone (5.6 g, 38 mmol), acetic acid (260 mL) and 4-5 drops of water was added sodium acetate (7.0 g, 83 mmol), followed by drop-wise addition of bromine (13.3 g, 83 mmol) in acetic acid (60 mL). The reaction mixture was stirred at room temperature for 18 hours, the resulting precipitate filtered, dried and collected. This gave 8.4 g (72%) of 4,6-dibromo-5-hydroxy-1-indanone as a solid mass which was used directly in the next step without further purification.

(c) To a stirred suspension under nitrogen consisting of bis-(3-isopropyl-4-methoxyphenyl)iodonium tetrafluoroborate (6.25 g, 12.2 mmol), copper bronze (1.02 g) and dichloro-methane (25 mL), was added a solution of 4,6-dibromo-5-hydroxy-1-indanone (2.50 g, 8.17 mmol) and triethylamine (1.00 g, 8.99 mmol) in dichloromethane (25 mL) at room temperature. The reaction mixture was stirred in the dark for 48 hours. The reaction mixture was filtered on a pad of celite, the filtrate concentrated and the residue purified on column (silica gel, n-heptane/ethyl acetate, gradient elution from 100 to 94% n-heptane). This gave 2.5 g (67%) of 3,5-dibromo-4-(3-isopropyl-4-methoxyphenoxy)-1-indanone.

(c) To a solution of 3,5-dibromo-4-(3-isopropyl-4-methoxyphenoxy)-1-indanone (2.00 mg, 4.4 mmol) solved in dry toluene (35 mL), was added zinkpowder (0.40 g, 5.9 mmol) followed by ethylbromo acetate (0.50 g, 2.9 mmol). The reaction mixture was heated at 130° C. and after 20 minutes a second portion of zinc powder (0.40 g, 5.9 mmol) and ethylbromo acetate (0.5 g, 2.9 mmol) was added. A third portion of zinc powder (0.40 g, 5.9 mmol) and ethylbromo acetate (0.5 g, 2.9 mmol) was added after 15 minutes. After 10 minutes the reaction mixture was cooled down to 0° C., water (75 mL) followed by hydrochloric acid (75 mL, 1 N) was added. The aqueous phase was extracted with ethyl acetate and the collected organic phases washed with water three times. The organic phase was dried over magnesium sulfate, concentrated and subjected to column (silica gel, n-heptane/ethyl acetate, gradient elution from 100 to 80% n-heptane). This gave 1.4 (72%) of ethyl[4,6-dibromo-1-hydroxy-5-(3-isopropyl-4-methoxyphenoxy)indane-1-yl]acetate.

(d) Triethylsilane (1.7 g, 15 mmol) followed by trifluoroacetic acid (40 mL) was added to ethyl [4,6-dibromo-1-hydroxy-5-(3-isopropyl-4-methoxyphenoxy)indane-1-yl]acetate (2.0 g, 3.7 mmol). The reaction mixture was stirred at room temperature and in nitrogen atmosphere for 5 hours. The reaction mixture was concentrated and purified on column (silica gel, n-heptane/ethyl acetate, gradient elution from 100 to 92% n-heptane), to give 1.6 g (82%) of ethyl[4,6-dibromo-5-(3-isopropyl-4-methoxyphenoxy)indane-1-yl]acetate.

(e) Boron trifluoride dimethyl sulfide complex (23 mL) was added at 0° C. to a solution of ethyl[4,6-dibromo-5-(3-isopropyl-4-methoxyphenoxy)indane-1-yl]acetate (2.0 g, 3.8 mmol) in dichloromethane (150 mL). The resulting reaction mixture was stirred under nitrogen atmosphere and at room temperature. After 16 hours, the reaction mixture was washed twice with brine, dried over magnesium sulfate and concentrated, to give 1.62 g (83%) of ethyl[4,6-dibromo-5-(3-isopropyl-4-hydroxyphenoxy)indane-1-yl]acetate.

(f) A mixture of ethyl[4,6-dibromo-5-(3-isopropyl-4-hydroxyphenoxy)indane-1-yl]acetate (20 mg, 0.039 mmol), potassium carbonate (11 mg, 0.080 mmol) and acetonitrile (0.75 mL) was stirred at room temperature for 30 minutes. 2-Bromomethylnapthalene (18 mg, 0.081 mmol) in acetonitrile (0.25 mL) was added and the reaction mixture was stirred at 80° C. for 16 hours. The reaction mixture was purified on a short column (SPE-silica, 1 g/6 mL, n-heptane/ethyl acetate 65:35), the filtrate concentrated and the resulting residue stirred with tetrahydrofuran (0.50 mL) and lithium hydroxide (0.5 mL, 1 N) at room temperature for 16 hours. The reaction mixture was filtered through a SCX-column (strong cation exchanger: benzenesulphonic acid silane, 1 g/3 mL, methanol as eluent) and the filtrate concentrated. The residue was dissolved in the smallest possible amount of dichloromethane and purified on two consequtive short columns (SPE-silica, 1 g/6 mL, n-heptane/ethyl acetate 9:1, and dichloro-methane/methanol 9:1). This gave 4.2 mg (17%) of {4,6-dibromo-5-[3-isopropyl-4-(naph-thalen-2-yl-methoxy)phenoxy]indan-1-yl}acetic acid. LC-MS (ES) m/z 623 (M−1).

### Example 2

### {4,6-Dibromo-5-[4-(4-fluorobenzyloxy)-3-isopropylphenoxy]indan-1-yl}-acetic Acid

4-Fluorobenzyl bromide (15 mg, 0.080 mmol) was coupled with ethyl[4,6-dibromo-5-(3-isopropyl-4-hydroxyphenoxy)indane-1-yl]acetate (20 mg, 0.039 mmol) and subsequently deprotected using the procedures as described in Example 1. This gave 5.3 mg (23%) of {4,6-dibromo-5-[4-(4-fluorobenzyloxy)-3-isopropylphenoxy]indan-1-yl}acetic acid. LC-MS (ES) m/z 591 (M−1).

### Example 3

### {4,6-Dibromo-5-[3-isopropyl-4-(5-methylisoxazol-3-ylmethoxy)phenoxy]-indan-1-yl}acetic Acid

A mixture of ethyl[4,6-dibromo-5-(3-isopropyl-4-hydroxyphenoxy)indane-1-yl]acetate (20 mg, 0.039 mmol), potassium carbonate (11 mg, 0.080 mmol) and acetonitrile (0.75 mL) was stirred at room temperature for 30 minutes. 3-Chloromethyl-5-methylisoxazole (10.5 mg, 0.080 mmol) and a catalytic amount of potassium iodide in acetonitrile (0.25 mL) was added, and the reaction mixture was stirred at 80° C. After 16 hours, the reaction mixture was purified on a short column (SPE-silica, 1 g/6 mL, n-heptane/ethyl acetate 65:35), the resulting filtrate concentrated and the resulting residue stirred with tetrahydrofuran (0.50 mL) and lithium hydroxide (0.5 mL, 1 N) at room temperature for 16 hours. The reaction mixture was filtered through a SCX-column (strong cation exchanger: benzenesulphonic acid silane, 1 g/3 mL, methanol as eluent) and the filtrate concentrated. The residue was dissolved in the smallest possible amount of dichloromethane and purified on two consequtive short columns (SPE-silica, 1 g/6 mL, n-heptane/ethyl acetate 9:1, and dichloromethane/methanol 9:1). This gave 7.0 mg (31%) of {4,6-dibromo-5-[3-isopropyl-4-(5-methylisoxazol-3-ylmethoxy)-phenoxy]indan-1-yl)acetic acid. LC-MS (ES) m/z 578 (M−1).

### Example 4

### {4,6-Dibromo-5-[3-isopropyl-4-(pyridin-2-yl-methoxy)phenoxy]indan-1-yl}acetic Acid

2-Picolyl chloride (10.2 mg, 0.080 mmol) was coupled with ethyl[4,6-dibromo-5-(3-isopropyl-4-hydroxyphenoxy)indane-1-yl]acetate (20 mg, 0.039 mmol) and subsequently deprotected using the procedures as described in Example 3. This gave 5.0 mg (22%) of {4,6-dibromo-5-[3-isopropyl-4-(pyridin-2-yl-methoxy)phenoxy]indan-1-yl}acetic acid LC-MS (ES) m/z 574 (M−1).

### Example 5

### {4,6-Dibromo-5-[3-isopropyl-4-(5-phenyl-[1,2,4]oxadiazol-3-ylmethoxy)-phenoxy]indan-1-yl}acetic Acid

3-Chloromethyl-5-phenyl[1,2,4]oxadiazole (15.6 mg, 0.080 mmol) was coupled with ethyl[4,6-dibromo-5-(3-isopropyl-4-hydroxyphenoxy)indane-1-yl]acetate and subsequently deprotected using the procedures as described in Example 3. This gave 11 mg (44%) of {4,6-dibromo-5-[3-isopropyl-4-(5-phenyl[1,2,4]-oxadiazol-3-ylmethoxy)phenoxy]indan-1-yl}acetic acid LC-MS (ES) m/z 641 (M−1).

### Example 6

### 4-[4-(4,6-Dibromo-1-carboxymethyl-indan-5-yloxy)-2-isopropylphenoxy-methyl]benzoic Acid

A mixture of ethyl[4,6-dibromo-5-(3-isopropyl-4-hydroxyphenoxy)indane-1-yl]acetate (20 mg, 0.039 mmol), potassium carbonate (11 mg, 0.080 mmol) and acetonitrile (0.75 mL) was stirred at room temperature for 30 minutes. Methyl 4-(bromomethyl)benzoate (20 mg, 0.080 mmol) in acetonitrile (0.25 mL) was added and the reaction mixture was stirred at 80° C. for 48 hours. The reaction mixture was purified on a short column (SPE-silica, 1 g/6 mL, n-heptane/ethyl acetate 65:35), the filtrate concentrated and the resulting residue stirred with tetrahydrofuran (0.50 mL) and lithium hydroxide (0.5 mL, 1 N) at room temperature for 16 hours. The reaction mixture was filtered through a SCX-column (strong cation exchanger: benzenesulphonic acid silane, 1 g/3 mL, methanol/water gradient from 0 to 50% methanol) and the filtrate concentrated. The residue was dissolved in dichloromethane and purified on two consequtive short columns (SPE-silica, 1 g/6 mL, n-heptane/ethyl acetate 9:1, and dichloromethane/methanol 9:1). This gave 8.0 mg (33%) of 4-[4-(4,6-dibromo-1-carboxy-methylindan-5-yloxy)-2-isopropylphenoxymethyl]benzoic acid LC-MS (ES) m/z 617 (M−1).

### Example 7

### (4,6-Dibromo-5-{4-[2-(1H-indol-2-yl)ethoxy]-3-isopropylphenoxy}indan-1-yl)acetic Acid

3-(2-Bromoethyl)indole-1-carboxylic acid tert-butyl ester (0.080 mmol) was coupled with ethyl[4,6-dibromo-5-(3-isopropyl-4-hydroxyphenoxy)indane-1-yl]acetate (20 mg, 0.039 mmol), and subsequently deprotected using the procedures as described in Example 6. This gave 11 mg (45%) of (4,6-dibromo-5-{4-[2-(1H-indol-2-yl)ethoxy]-3-isopropylphenoxy}-indan-1-yl)acetic acid LC-MS (ES) m/z 626 (M−1).

### Example 8

### (4,6-Dibromo-5-[3-isopropyl-4-(5-thiophen-3-yl-[1,2,4]oxadiazol-3-yl-methoxy)phenoxy]indan-1-yl}acetic Acid

A mixture of ethyl [4,6-dibromo-5-(3-isopropyl-4-hydroxyphenoxy)indane-1-yl]acetate (20 mg, 0.039 mmol), potassium carbonate (20 mg, 0.14 mmol) and acetonitrile (0.75 mL) was stirred at room temperature for 30 minutes. 3-Chloromethyl-5-thiophen-3-yl-[1,2,4]-oxadiazole (16 mg, 0.080 mmol) and a catalytic amount of potassium iodide in acetonitrile (0.25 mL) was added, and the reaction mixture was stirred at 80° C. After 16 hours, the reaction mixture was purified on a short column (SPE-silica, 1 g/6 mL, n-heptane/ethyl acetate 65:35), the resulting filtrate concentrated and the resulting residue stirred with tetrahydrofuran (0.50 mL) and lithium hydroxide (0.5 mL, 1 N) at room temperature for 16 hours. The reaction mixture was filtered through a SCX-column (strong cation exchanger: benzenesulphonic acid silane, 1 g/3 mL, methanol as eluent) and the filtrate concentrated. The residue was purified on hplc (ZorBax SBC8, acetonitrile/water/formic acid, gradient elution during 15 minutes starting at 5:95:0.1 and ending at 100:0:0.1, λ=254 nm), to give 0.8 mg (3.2%) of (4,6-dibromo-5-[3-isopropyl-4-(5-thiophen-3-yl-[1,2,4]oxadiazol-3-yl-methoxy)-phenoxy]indan-1-yl}acetic acid LC-MS (ES) m/z 647 (M−1).

### Example 9

### {5-[4-(4-Amino-6-phenylamino[1,3,5]triazin-2-ylmethoxy)-3-isopropyl-phenoxy]-4,6-dibromoindan-1-yl}acetic Acid

6-Chloromethyl-N-phenyl[1,3,5]triazine-2,4-diamine (19 mg, 0.080 mmol) was coupled with ethyl[4,6-dibromo-5-(3-isopropyl-4-hydroxyphenoxy)indane-1-yl]acetate (20 mg, 0.039 mmol), and subsequently deprotected using the procedures as described in Example 8. This gave 13 mg (50%) of {5-[4-(4-amino-6-phenylamino[1,3,5]triazin-2-ylmethoxy)-3-isopropyl-phenoxy]-4,6-dibromoindan-1-yl}acetic acid LC-MS (ES) m/z 682 (M−1).

### Example 10

### {4,6-Dibromo-5-[3-isopropyl-4-(5-methyl-2-phenyloxazol-4-ylmethoxy)-phenoxy]indan-1-yl}acetic Acid

A mixture of ethyl[4,6-dibromo-5-(3-isopropyl-4-hydroxyphenoxy)indane-1-yl]acetate (20 mg, 0.039 mmol), potassium carbonate (20 mg, 0.14 mmol) and acetonitrile (0.75 mL) was stirred at room temperature for 30 minutes. 4-Chloromethyl-5-methyl-2-phenyloxazole (16 mg, 0.080 mmol) and a catalytic amount of potassium iodide in acetonitrile (0.25 mL) was added, and the reaction mixture was stirred for 60 hours at room temperature. The reaction mixture was purified on a short column (SPE-silica, 1 g/6 mL, n-heptane/ethyl acetate 65:35), the resulting filtrate concentrated and the resulting residue stirred with tetrahydrofuran (0.50 mL) and lithium hydroxide (0.5 mL, 1 N) at room temperature for 16 hours. The reaction mixture was filtered through a SCX-column (strong cation exchanger: benzenesulphonic acid silane, 1 g/3 mL, methanol as eluent) and the filtrate concentrated. The residue was purified on hplc (ZorBax SBC8, acetonitrile/water/fommic acid, gradient elution during 15 minutes starting at 5:95:0.1 and ending at 100:0:0.1, λ=254 nm), to give 18 mg (70%) of (4,6-dibromo-5-[3-isopropyl-4-(5-thiophen-3-yl-[1,2,4]oxadiazol-3-yl-methoxy)-phenoxy]-indan-1-yl}acetic acid LC-MS (ES) m/z 654 (M−1).

### Example 11

### {4,6-Dibromo-5-[4-(3,5-dimethylisoxazol-4-ylmethoxy)-3-isopropyl-phenoxy]indan-1-yl}acetic Acid

4-Chloromethyl-3,5-dimethylisoxazol (12 mg, 0.080 mmol) was coupled with ethyl[4,6-dibromo-5-(3-isopropyl-4-hydroxyphenoxy)indane-1-yl]acetate (20 mg, 0.039 mmol), and subsequently deprotected using the procedures as described in Example 10. This gave 14 mg (60%) of {5-[4-(4-amino-6-phenylamino[1,3,5]triazin-2-ylmethoxy)-3-isopropylphenoxy]-4,6-dibromoindan-1-yl}acetic acid LC-MS (ES) m/z 592 (M−1).

The compounds of the invention exhibit binding affinities to the ThRα receptor in the range of 100 to 500 nM.

1. A compound according to the general formula 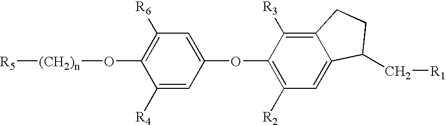
or a pharmaceutically acceptable salt thereof, wherein:
R₁ is independently selected from: carboxylic acid (—CO₂H); phosphonic acid (—PO(OH)₂); phosphamic acid (—PO(OH)NH₂); sulphonic acid (—SO₂OH); hydroxamic acid (—CONHOH); oxamic acid (—NHCOCO₂H); and malonamic acid (—NHCOCH₂CO₂H), or any other possible bioisosteric equivalent of the groups above;
R₂ and R₃ are the same or different and independently selected from: chlorine; bromine; iodide; C₁₋₄ alkyl, said alkyl, or a bioisosteric equivalent optionally substituted with 0, 1, 2 or 3 groups of R^{a} which groups may be the same or different;
R₄ and R₆ are the same or different and independently selected from: hydrogen; halogen; C₁₋₄ alkyl; or a bioisosteric equivalent optionally substituted with 0, 1, 2 or 3 groups of R^{a} which groups may be the same or different;
R₅ is selected from: C₆₋₁₀ aryl; C₁₋₉ heteroaryl, said aryl;
and heteroaryl optionally substituted with 0, 1, 2 or 3 groups of R^{b} which groups may be the same or different;
R^{a} represents fluorine or chlorine;
R^{b} represents a member selected from the group of: halogen; —CN; —CO₂H; —CHO; —NH₂; C₁₋₄ alkyl; C₂₋₄ alkenyl; C₂₋₄ alkynyl; C₁₋₄ alkoxy; C₂₋₄ alkenoxy; C₂₋₄ alkynoxy; C₁₋₄ alkylthio; C₂₋₄ alkenylthio; C₂₋₄ alkynylthio; C₆ aryl; C₁₋₅ heteroaryl; C₃₋₆ cycloalkyl; —NH(C₁₋₄); —N(C₁₋₄)₂; —NH(C₆aryl); —N(C₆aryl)₂; —NH (C₁₋₅ heteroaryl); and —N(C₁₋₅ heteroaryl)₂ or a bioisosteric equivalent;
n is an integer of 1, 2 or 3;
included for the variables above are all the possible stereoisomers thereof; prodrug ester forms thereof; and
radioactive forms thereof.

2. A compound according to claim 1 wherein R₁ is carboxylic acid (—CO₂H).

3. A compound according to claim 1 wherein R₂ and R₃ is bromine or chlorine.

4. A compound according to claim 1, wherein R₄ is isopropyl and R₆ is hydrogen.

5. A compound according to claim 1, wherein R₂ and R₃ is bromine.

6. A compound according to claim 1, which is:
{4,6-Dibromo-5-[3-isopropyl-4-(naphthalen-2-yl-methoxy)phenoxy]indan-1-yl}-acetic acid;
{4,6-Dibromo-5-[4-(4-fluorobenzyloxy)-3-isopropylphenoxy]indan-1-yl}acetic acid;
{4,6-Dibromo-5-[3-isopropyl-4-(5-methylisoxazol-3-ylmethoxy)phenoxy]indan-1-yl}acetic acid;
{4,6-Dibromo-5-[3-isopropyl-4-(pyridin-2-yl-methoxy)phenoxy]indan-1-yl}acetic acid;
4,6-Dibromo-5-[3-isopropyl-4-(5-phenyl-[1,2,4]oxadiazol-3-ylmethoxy)phenoxy]-indan-1-yl}acetic acid;
4-[4-(4,6-Dibromo-1-carboxymethyl-indan-5-yloxy)-2-isopropylphenoxymethyl]-benzoic acid;
(4,6-Dibromo-5-4-[2-(1H-indol-2-yl)ethoxy]-3-isopropylphenoxy}indan-1-yl)acetic acid;
(4,6-Dibromo-5-[3-isopropyl-4-(5-thiophen-3-yl-[1,2,4]oxadiazol-3-yl-methoxy)-phenoxy]indan-1-yl}acetic acid;
{5-[4-(4-Amino-6-phenylamino[1,3,5]triazin-2-ylmethoxy)-3-isopropylphenoxy]-4,6-dibromoindan-1-yl}acetic acid;
{4,6-Dibromo-5-[3-isopropyl-4-(5-methyl-2-phenyloxazol-4-ylmethoxy)phenoxy]-indan-1-yl}acetic acid;
{4,6-Dibromo-5-[4-(3,5-dimethylisoxazol-4-ylmethoxy)-3-isopropylphenoxy]-indan-1-yl}acetic acid;
and pharmaceutically acceptable salts thereof, and stereoisomers thereof.

7. A compound according to claim 1, which have one or more asymmetric centers and can exist in the form of racemates, single and multiple enantiomers, as individual diastereomers, with all possible isomers, and mixtures thereof.

8. A compound according to claim 1, for use in medical therapy.

9. A pharmaceutical composition comprising an effective amount of a compound according to claim 1, or a pharmaceutically effective salt thereof, together with a pharmaceutically acceptable carrier.

10. A method for preventing, inhibiting or treating a disease which is dependent on the expression of a T₃ regulated gene or associated with metabolic dysfunction, which comprises administering to a patient in need of treatment a therapeutically effective amount of a compound according to claim 1.

11. The method according to claim 10 wherein the disease is selected from cardiac arrythmias, thyrotoxicosis, subclinical hyperthyrodis, certain skin disorders, and certain liver diseases.

12. The method according to claim 11 wherein the disease is a skin disorder or skin disease.

13. The method according to claim 12 wherein the skin disorder or skin disease is selected from: keloids, lichen planus, ichtyosis, acne, psoriasis, Dernier's disease, eczema, atopic dermatitis, chloracne, pityriasis, and hirsuitism.

14. The method according to claim 11 wherein the disease is a liver disorder or liver disease.

15. The method according to claim 14 wherein the liver disorder or liver disease is selected from: chronic alcoholism, acute hepatitis, hepatitis C-induced liver cirrhosis, and liver fibrosis.

16. A method to treat skin disorders or diseases comprising the step of administering to a patient suffering skin disorders or diseases a pharmaceutical composition comprising a compound according to claim 1 in a combination with a retoid or a Vitamin D analog.

17-24. (canceled)
